(19) United States
(12) Patent Application Publication
(10) Pub. No.: US 2026/0165673 A1
(43) Pub. Date: Jun. 18, 2026

(54) PORTABLE ULTRASOUND DEVICE

(71) Applicant: Kimberly Ballard Matlock, Keller, TX (US)

(72) Inventor: Kimberly Ballard Matlock, Keller, TX (US)

(21) Appl. No.: 19/532,026

(22) Filed: Feb. 6, 2026

Related U.S. Application Data
(63) Continuation of application No. PCT/US2025/038863, filed on Jul. 23, 2025.
(60) Provisional application No. 63/676,214, filed on Jul. 26, 2024.
(60) Provisional application No. 63/725,268, filed on Nov. 26, 2024.

(51) Int. Cl. A61B 8/08; A61B 8/00

(52) U.S. Cl. CPC A61B 8/085; A61B 8/461; A61B 8/54

(57) ABSTRACT

Embodiments of an ultrasound medical device and methods of determining an overall constipation score are disclosed. The device comprises a power source, a control unit coupled to the power source, a display in electrical communication with the control unit, a transducer in communication with the control unit, a memory coupled to the control unit, the memory having instructions for recording ultrasound data during a scanning session and determining an overall constipation score using various factors determined from the scanning session.

### CROSS-REFERENCE TO RELATED APPLICATIONS

This application is a U.S. continuation of the International Application No. PCT/US2025/038863, filed on Jul. 23, 2025, entitled “PORTABLE ULTRASOUND DEVICE,” which claims the benefit of the filing date of U.S. provisional patent application Ser. No. 63/676,214, filed on Jul. 26, 2024, entitled “IMPROVED PORTABLE ULTRASOUND DEVICE,” and claims the benefit of the filing date of U.S. provisional patent application Ser. No. 63/725,268, filed on Nov. 26, 2024, entitled “IMPROVED PORTABLE ULTRASOUND DEVICE,” all of the above disclosures are incorporated herein by reference for all purposes.

### TECHNICAL FIELD

The invention relates in general to medical ultrasound scanning machines and, in particular, to automated ultrasound scanning machines designed to display a constipation score.

### BACKGROUND INFORMATION

Constipation is a common gastrointestinal issue affecting individuals of all ages, with prevalence rates ranging between 12% and 30% worldwide. The disorder is characterized by infrequent, difficult, or incomplete bowel movements, often causing discomfort, pain, encopresis, and, in severe cases, significant medical complications. Constipation can be caused by a range of factors, including dietary habits, lifestyle, medication use, genetic tendencies, and underlying health conditions.

Accurate diagnosis of constipation, however, presents significant challenges in clinical settings. Diagnostic assessments for constipation typically rely on self-reported symptoms, questionnaires, physical exams, and x-ray imaging. Current methods for diagnosing constipation often lack accuracy. Patients may inaccurately report symptoms, and certain signs of constipation may only become evident at an advanced stage. X-ray interpretation of constipation is subjective, and clinical guidelines vary. The absence of a standard, objective diagnostic method can delay the identification of constipation, especially in early or asymptomatic cases.

Due to the limited accuracy and inconvenience of current diagnostic methods, the ability of healthcare providers to identify and treat constipation effectively is not optimal. Consequently, there is a growing need for improved diagnostic tools that can provide objective, reliable, and non-invasive assessments of constipation, thereby enabling timely intervention and improved patient outcomes. What is needed, therefore, is a device that can objectively and accurately identify and quantify the level of constipation in a patient in a safe and efficient way.

### SUMMARY

In one embodiment, an ultrasound medical device is presented, and methods of determining an overall constipation score are disclosed. In some embodiments, the device utilizes the patient's bladder as an “acoustic window” to determine the stool quality and location. In certain embodiments, the device comprises a power source, a control unit coupled to the power source; a display in electrical communication with the control unit; a transducer in communication with the control unit, a memory coupled to the control unit, the memory having instructions for recording ultrasound data during a scanning session and determining an overall constipation score using various external factors, such as the patients age or size, and factors determined from the scanning session as discussed below.

In another embodiment, there is disclosed a handheld battery-operated ultrasound device that has Artificial Intelligence (“AI”) software technology to determine an overall constipation score. This constipation score corresponds to the presence and severity of constipation. The ultrasound device may be placed approximately 2 cm above the pubic synthesis and scanned to the xiphoid process. Scans may be performed in both the transverse and longitudinal planes.

In one embodiment, after the patient's age range or size is input, the ultrasound device detects stool quality, whether standard, retained (i.e., half moon shape), or hard (i.e., crescent shape). The ultrasound device then detects the apex (stool height in the abdomen) of this crescent shape and may use this location as a factor or value to contribute to an overall constipation score. The ultrasound device then determines the effects of the fecal mass on the bladder and assigns that effect a predetermined value. This contributes to the constipation score. Another factor may be the rectal diameter which is an indicator of the patient's age or size. In certain embodiments, the ultrasound device measures rectal diameter and assigns a predetermined value based on the diameter. Another factor may be stool quality. Once such factors have been determined, the ultrasound device (or a computer in communication with the ultrasound device) uses these values to calculate a total constipation score to arrive at an objective indication of the degree of constipation for a patient.

Embodiments also relate to a medical ultrasound device that includes a power source, a control unit coupled to this power source, a display in electrical communication with the control unit, a transducer connected to the control unit, and a memory interfaced with the control unit. The memory contains instructions for recording ultrasound data obtained during a scanning session and for determining various effect parameters of the patient, such as stool height, bladder status, rectal diameter, and stool quality, all derived from the ultrasound data. Based on these parameters, the device calculates an overall constipation score for the patient, which is then displayed on the device's screen. The stool height effect parameter is determined by assigning a value based on the stool's position relative to the patient's bladder and umbilicus. The bladder effect parameter is assigned a value depending on the bladder's state, which can include being empty, non-compressed, indented, or displaced. The stool quality effect parameter is evaluated by assigning a value depending on whether the stool is normal, retained, or hard. Furthermore, the control unit is capable of communicating with an artificial intelligence system that includes a neural network trained for recognizing anatomical structures within the torso from ultrasound images. This AI system can be supplemented by a training module designed to identify relevant anatomical features within collected ultrasound data.

These and other features, and advantages, will be more clearly understood from the following detailed description taken in conjunction with the accompanying drawings. It is important to note the drawings are not intended to represent the only aspect of the invention. The features and advantages of the present disclosure will be readily apparent to those skilled in the art. While numerous changes may be made by those skilled in the art, such changes are within the spirit of the invention.

### BRIEF DESCRIPTION OF THE DRAWINGS

FIG. 1. is a functional diagram illustrating one embodiment of an ultrasound imaging device that incorporates one or more aspects of the present invention.

FIG. 2 is a functional diagram of a transducer that may be incorporated into various aspects of the present invention.

FIG. 3A is a perspective view of one embodiment of a handheld ultrasound device incorporating various aspects of the present invention. FIG. 3B is a perspective view of the device of FIG. 3A, with a portion of the cover removed for clarity.

FIG. 4 is an example process flow diagram illustrating a method for determining an overall constipation score which may be used by various embodiments of the present invention.

FIG. 5 is an example process flow diagram illustrating a method for determining a stool height parameter, which may be used by various embodiments of the present invention.

FIG. 6 is an example process flow diagram illustrating a method for determining a bladder effect parameter, which may be used by various embodiments of the present invention.

FIG. 7 is an example process flow diagram illustrating a method for determining a rectal diameter parameter, which may be used by various embodiments of the present invention.

FIG. 8 is an example process flow diagram illustrating a method for determining a stool quality parameter that may be used by various embodiments of the present invention.

### DETAILED DESCRIPTION

Specific examples of components, signals, messages, protocols, and arrangements are described below to simplify the present disclosure. These are, of course, merely examples and are not intended to limit the invention from that described in the claims. Well-known elements are presented without detailed descriptions in order not to obscure the present invention in unnecessary detail. For the most part, details unnecessary to obtain a complete understanding of the present invention have been omitted in as much as such details are within the skills of persons of ordinary skill in the relevant art. Details regarding control circuitry or mechanisms used to control the function of the various elements described herein are omitted, as such control circuits are within the skills of persons of ordinary skill in the relevant art.

When directions, such as upper, lower, top, bottom, clockwise, or counter-clockwise, are discussed in this disclosure, such directions are meant to only supply reference directions for the illustrated figures and orientation of components with respect to each other or to illustrate the figures. The directions should not be read to imply actual directions used in any resulting invention or actual use. Under no circumstances should such directions be read to limit or impart any meaning to the claims.

### System Overview

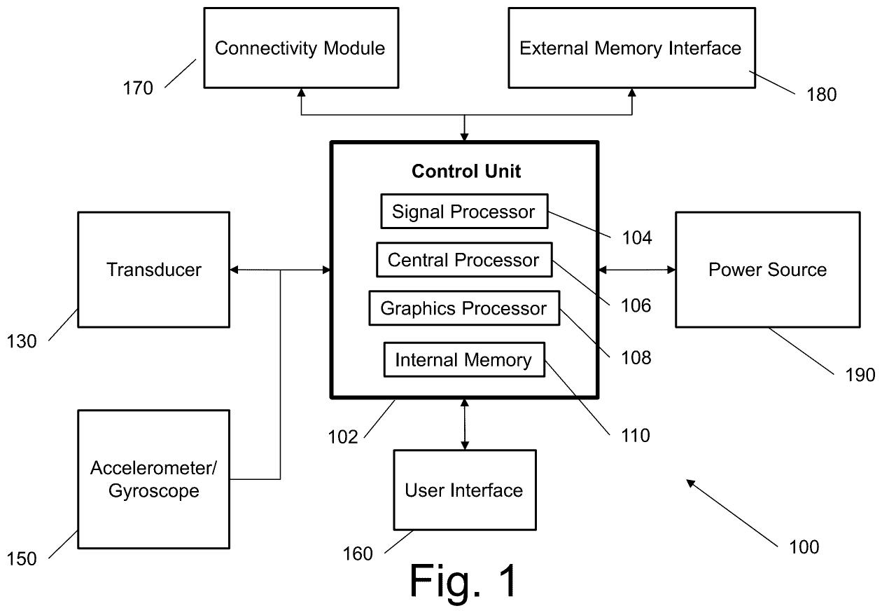

Turning now to FIG. 1, there is presented a functional diagram of one embodiment of a portable ultrasound device 100. The disclosed embodiment is a portable handheld ultrasound device and is designed to be compact, user-friendly, and efficient for point-of-care constipation diagnostics. In certain embodiments, the major functional components of this device 100 may include: a transducer control unit or controller 102, a transducer 130; an accelerometer and/or gyroscope 150; a user interface 160; a connectivity module 170; an external memory interface 180; and a power source 190.

In certain embodiments, the control unit 102 may include a signal processor 104 for processing the signals received from the transducer 130. In certain embodiments, the control unit 102 may include one or more central processors 106 for controlling the overall functions of the device 100, and optionally, an image or graphics processor 108 for converting the processed signals into a visual image; and some form of internal memory 110 for storing software and data received from the transducer and sensors. In certain embodiments, the software includes functionality such as image capture, storage, communication, and analysis.

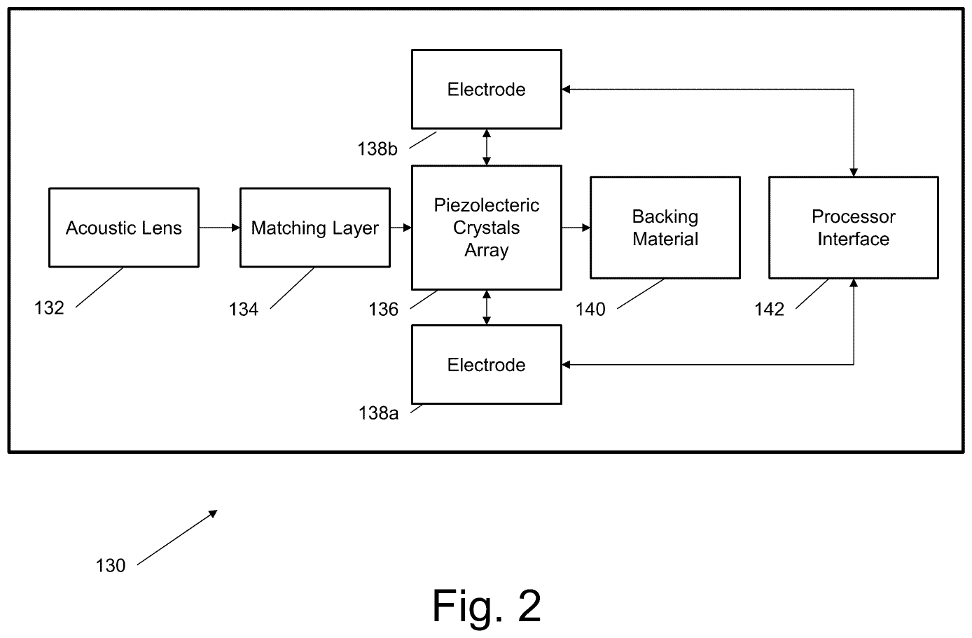

As will be described in detail below in relation to FIG. 2, the transducer or probe 130 converts electrical signals into sound waves and vice versa. The transducer 130 is responsible for generating and receiving ultrasound waves to create images of internal body structures. Certain embodiments may use one or more sensors, such as an accelerometer and gyroscope 150, for orientation detection and user interface adjustments based on the relative position of the device to the patient's anatomy. Software running in the control unit 102 may track the position of the device 100 relative to the patient's anatomy so that the processed sound signals can be stitched together to generate data or a database which could represent or create a mosaic and/or an overall visual image of a portion of the patient's anatomy.

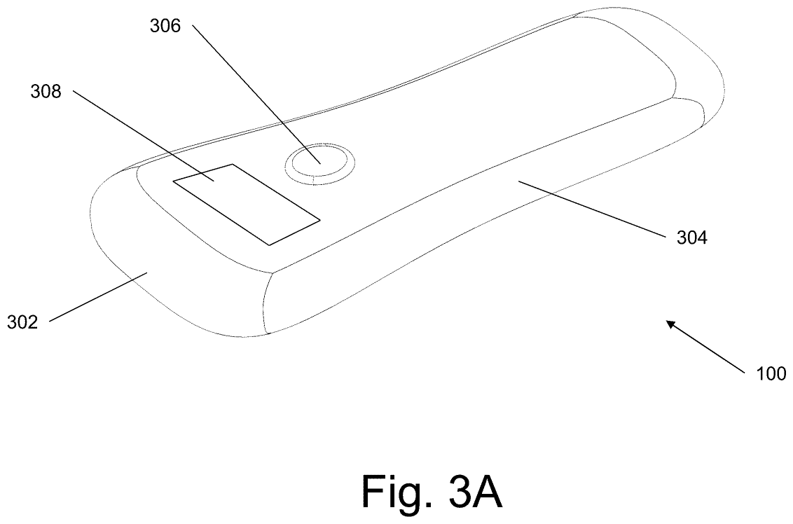

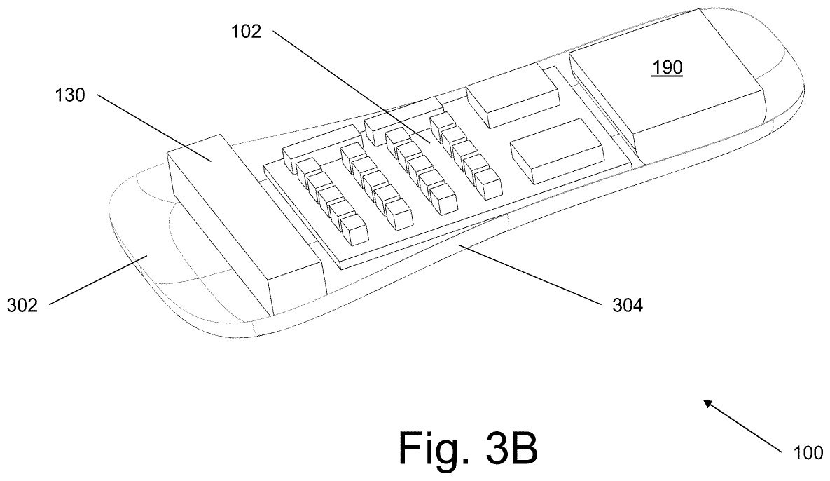

In certain embodiments, there may be a primary or user interface 160 on the device 100 itself such as a primary or main control button 306 and/or a small flat touch-screen display 308 (See FIG. 3A). As illustrated in FIG. 3A discussed below, in certain embodiments, there may be a main control button 306 for turning the device on and off and to indicate when to start and stop the scanning process. In some embodiments, there may also be a screen 308, such as an LED or LCD screen, for displaying an overall constipation score and/or for allowing the user to customize certain system settings.

In certain embodiments, basic system settings and patient information may be input through the primary button 306 or a microphone (not shown), which allows the control unit 102 to accept voice commands. In certain embodiments, patient information such as a patient's size and age and system settings such as sensitivity, directional power, adjusting gain, adjusting frequency, scan depth, wall filter adjustments, mode trace, sound on or off, volume, setting time and date may be entered via an application running on a processor in wireless or wired communication with connectivity module 170 discussed below. In yet other embodiments, there may be additional or larger displays, physical buttons, sounds, and/or touchscreen controls for inputting the patient's information, such as age or size, and adjusting or inputting system settings like gain, scanning depth, and mode (e.g., B-mode, Doppler). Again, such settings may also be set via an associated software application residing on a separate computing device, such as a phone or tablet (not shown), running an app designed to communicate and control specific processes within the device 100.

In certain embodiments, there may be a speaker (not shown) for audio output, such as Doppler sounds, warning, or confirmation signals. As mentioned above, certain embodiments may include a microphone for inputting voice instructions and/or voice annotations.

In certain embodiments, the connectivity module 170 includes wireless connectivity, such as Bluetooth or Wi-Fi, for connecting to other devices, cloud storage, or networks. Certain embodiments may also have data transfer ports such as USB and HDMI ports for wired connections to computers, handheld computers, tablets, larger displays, or external storage devices.

Some embodiments may have an external memory or a storage interface 180, such as support for external memory cards, such as SD cards, for additional storage and data transfer capabilities, such as a USB port.

In certain embodiments, there may be a portable power source 190, such as a rechargeable battery, to provide power for portable use. The power source 190 may also be coupled to a charging port for recharging the battery via USB-C or other proprietary chargers.

### Overview of an Example Transducer

Turning now to FIG. 2, there is illustrated a conceptual view of an example transducer 130 that may be used in certain embodiments. Such an example transducer 130 may include an acoustic lens 132, which focuses an ultrasound beam to improve spatial resolution and image quality. Such lenses are often made of silicone rubber or other flexible materials with suitable acoustic properties known in the art.

Certain embodiments may also include a matching layer 134 designed to minimize the acoustic impedance mismatch between a piezoelectric crystal array 136 and the patient's body, improving the efficiency of sound wave transmission into the body. The matching layer 134 may be made of a material with intermediate acoustic properties between the piezoelectric crystal array 136 and the tissues of the patient (not shown).

The piezoelectric crystal array 136 converts electrical energy into sound waves during transmission and sound waves back into electrical energy during reception. The piezoelectric crystal array may be formed from materials such as lead zirconate titanate (PZT), which have strong piezoelectric properties.

In certain embodiments, electrodes 138a and 138b apply the electrical signals to the piezoelectric crystal array 136 and receive the electrical signals generated by the returning echoes. In certain embodiments, the electrodes 138a and 138b may be placed on either side of the piezoelectric crystal array 136.

Certain embodiments may utilize a backing material 140 that dampens the vibrations of the piezoelectric crystals in the piezoelectric crystal array to stop ringing and produce short pulses, which improves axial resolution. The backing material 140 may be formed from a dense, attenuative material, such as epoxy resin loaded with tungsten or other heavy metals.

Certain embodiments include a processor interface 142, which may have connectors and cables for transmitting electrical signals between the transducer and the control unit or processor of the ultrasound machine. Such cables may include multiple coaxial cables or twisted pairs, shielded to prevent electromagnetic interference. In certain embodiments, there may be connectors to couple the transducer cables to the ultrasound machine's control unit.

### An Example Housing or Chassis

Turning now to FIGS. 3A and 3B, there is illustrated an example handheld device 100 which is designed to be compact and lightweight for ease of use. FIG. 3A is a perspective view of the handheld UV device 100. FIG. 3B is the perspective view of FIG. 3A, but with certain portions of a housing 304 removed for clarity.

As illustrated in FIG. 3B, the transducer 130 may be positioned at or close to a distal end 302 of the handheld device 100. In certain embodiments, the processor interface 142 (not shown) electronically couples to the power source 190 and the control unit 102. In other embodiments, the transducer 130 may be powered through the control unit 102. In either case, the control unit 102 has the ability to control the power output of the transducer 130.

In the illustrative embodiment, the control unit 102 is physically positioned between the transducer 130 and the power source 190. In certain embodiments, the housing or portions of the housing 304 surrounding the transducer 130 may have acoustical insulation (not shown) strategically placed within the housing to prevent the escape of ultrasound energy in undesired directions and to minimize internal reflections and noise.

In certain embodiments, the housing 304 may also encase heat sinks and/or fans (not shown) to assist the internal components in remaining relatively cool during operation.

As illustrated in FIG. 3A, in certain embodiments, there may be a main button 306 that functions as one of the primary user interfaces during scanning. For instance, a long button press (over 5 seconds) functions as an “on/off toggle” switch. Once the device is powered on, a short press of the main button indicates that the user is beginning the scanning process, and a following short press indicates that the user has ended the scanning process. A double press may indicate to the system that the user is about to start a new scan.

In certain embodiments, a flat screen, such as an LCD or LED screen 308, may be used to display an overall constipation score. When the screen is not displaying the overall constipation score, the screen can also be used to confirm modes indicated by the button presses discussed above.

In certain embodiments, the housing 304 may include a textured portion (not shown) and be ergonomically designed for comfortable handling during use. In certain embodiments, the housing 304 may be made from durable plastic or composite materials designed to withstand repeated use and disinfection.

### Central Processor and AI Integration

As will be explained in more detail below, the central processor 106 may analyze and display a constipation score based on the data or images (i.e., scans) received from the scanning process. Certain embodiments may utilize the patient's bladder as an acoustic window, in conjunction with advanced AI techniques, to assess the patient's stool quality and location. This allows the analysis of images and data to be more accurate and efficient. For example, an AI training process designed to recognize relevant anatomical structures within ultrasound scans is described below:

In certain embodiments, a comprehensive data collection phase involves gathering a large and diverse dataset of ultrasound images. These images should encompass a wide range of anatomical features, including structures such as the bladder, umbilicus, small and large intestines, rectum, and the presence and characteristics of stool within the intestines. The dataset may include images captured from different patients, varying angles, depths, and imaging conditions to ensure model robustness. This dataset enables the AI system to understand and localize specific structures which is helpful for assessing bowel conditions and device positioning within the patient's anatomy.

To enhance the annotation accuracy of the dataset, trained medical experts can manually label or annotate key features within each ultrasound image used in the AI training set. This process involves marking structures such as the bladder, intestines, rectum, stool, and other relevant tissues or features like fluid levels or bowel wall characteristics. Accurate annotations enable supervised learning, allowing the AI model to learn the precise recognition of targeted features.

Preprocessing steps ensure consistency and quality of the data: images are normalized to a standard pixel value range (e.g., 0-1 or 0-255) to minimize variability across datasets. Data augmentation techniques, such as random rotations, flips, zooms, cropping, and intensity variations, can be applied to artificially enlarge the dataset and improve model generalization, especially when limited real-world data is available.

Segmentation masks may be created to delineate specific regions within the images, such as stool patches, intestinal walls, or other anatomical features. These masks facilitate segmentation tasks, enabling the AI model to focus on identifying and quantifying aspects such as stool height, size, or shape, which can correlate with constipation severity.

After comprehensive preprocessing, an appropriate AI architecture may be selected based on the task: classification, segmentation, or a combination. Typical architectures for medical image analysis include Convolutional Neural Networks (CNNs) for classification tasks (e.g., determining the presence or absence of stool accumulation) and U-Net or Mask R-CNN architectures for image segmentation (e.g., isolating stool regions or anatomical boundaries). These models can be implemented from scratch or adapted from existing frameworks.

To accelerate development and improve performance, transfer learning techniques may be employed—using pre-trained models such as those trained on large datasets like ImageNet—and subsequently fine-tuning them on the specific ultrasound dataset. This approach leverages learned features that can be adapted to medical imaging tasks, reducing training time and improving accuracy.

The dataset is divided into training, validation, and testing subsets to objectively evaluate model performance. The training set is used to optimize the model parameters, while the validation set guides hyperparameter tuning, such as learning rate, number of layers, or regularization coefficients. Loss functions tailored to the task are utilized: cross-entropy loss for classification tasks and Dice coefficient or Intersection over Union (IoU) for segmentation. Optimization algorithms, such as Adam or stochastic gradient descent (SGD), are employed to minimize the loss and refine the model weights.

Model evaluation involves assessing performance metrics suited to the task—accuracy, precision, recall, F1 score for classification, and IoU or Dice coefficient for segmentation. These metrics help quantify how well the model identifies relevant features, segments regions accurately, and generalizes to unseen data.

Additional considerations may include addressing class imbalance issues—if certain stool types or anatomical features are underrepresented, techniques such as oversampling, undersampling, or weighted loss functions may be applied to prevent biased predictions. Continuous model improvement can be achieved by monitoring its performance in real-world clinical settings and periodically retraining with new data, thereby enhancing accuracy and ensuring robustness over time.

To implement these processes, various tools and libraries may be are employed, including deep learning frameworks like TensorFlow, Keras, or PyTorch. Medical imaging-specific libraries such as SimpleITK or OpenCV support image preprocessing and visualization. Annotation tools like Labelbox, VGG Image Annotator (VIA), or ITK-SNAP aid in creating precise labeled datasets necessary for supervised learning.

The AI development methodology described above serves as one exemplary approach; however, other AI techniques, including semi-supervised learning, reinforcement learning, or advanced architectures, may also be used within the scope of the present invention to improve model performance and adaptability.

### The Scanning Process

The manner of using one embodiment of the handheld device 100 will now be described. In certain embodiments, the device uses the patient's bladder as an acoustic window. For purposes of this application, an acoustic window is the optimal pathway through which ultrasound waves can pass most effectively to visualize the stool in a patient's body. The acoustic window is typically a region with minimal interference (such as bone, gas, or dense tissues) that allows clear transmission of sound waves and imaging.

To begin the process, the user applies an appropriate amount of ultrasound gel to the transducer or distal end 302 to eliminate air gaps and improve the transmission of sound waves. The user places the transducer 2 cm above the pubic symphysis while using the interface (primary button) to instruct the transducer 130 to start sending and receiving sound waves. The user should ensure that the transducer is correctly aligned and tilted caudally, maintaining steady skin contact. The device is then moved upward while scanning until it reaches the xyphoid process, adjusting the transducer's angle (relative to the patient) as needed to capture the necessary views of the organs. Longitudinal data and/or images are obtained next with the scanner probe again placed 2 cm above the pubis and tilted caudally, then the user moves the device 100 upward while scanning from the pubis to the Xyphoid process.

In certain embodiments, the user presses the primary button 306 (see FIG. 3A) once to start the scanning process. The user presses the button 306 again upon reaching the umbilicus, signaling the device to continue or adjust processing. Once the device reaches the xyphoid process, another press of the button 306 signals to the unit to calculate the overall height of the area.

During the scanning process, the control unit 102 receives signals from the user interface 160 (e.g., button 306) to start or continue scanning and sends electrical pulses to the transducer 130. The piezoelectric crystals 136 convert these electrical pulses into ultrasound waves, with the matching layer 134 ensuring efficient transmission into the body.

Ultrasound waves reflect off tissues and structures within the body and return to the transducer 130. The returning waves cause the piezoelectric crystals 136 to vibrate, generating electrical signals. These signals are captured by the electrodes 138a and 138b and sent back to the signal processor 104 of the control unit 102. The focus of the tissues and structure in the scans includes the stool in the intestine, bladder, and rectum. In certain embodiments, other abdominal organs are not analyzed, except in some cases for confirming position and alignment.

Sensors (not shown) determine the overall position of the transducer 130 with respect to the patient's anatomy and may use previously stored images and other AI data to ensure accurate alignment. Additional signals from the transducer 130 are received and processed by the signal processor 104, which passes the received signals to the primary central processor 106. The primary central processor 106 analyzes the signals and stores the data and analysis in the internal memory 110.

### Central Processor/Software Application

As discussed above, once the AI model is created, the AI model may be integrated into software or firmware for automatic measurements and diagnostics. In certain embodiments, the application software may reside in the internal memory 110 (see FIG. 1) coupled to the main processor(s) 106 within the handheld device 100. In other embodiments, there may be additional application programs residing in the memories of another computing device, such as a smartphone, tablet, or desktop computer (not shown), which also has an interface for configuring and/or controlling the device 100. Some of the features that may be offered by the application and/or software are described below.

In certain embodiments, the software may have various applications and features enabled by imaging modes such as B-mode (brightness), M-mode (motion), and Doppler (blood flow).

In some embodiments where an image is transferred to another device for viewing, there may be additional processing tools for enhancing image quality, measuring dimensions, and annotating.

Once image data is obtained via hardware and the scanning process discussed above, an overall constipation score may be calculated and displayed on the handheld device itself or, in some embodiments, on the display of another computing device in communication with the handheld device.

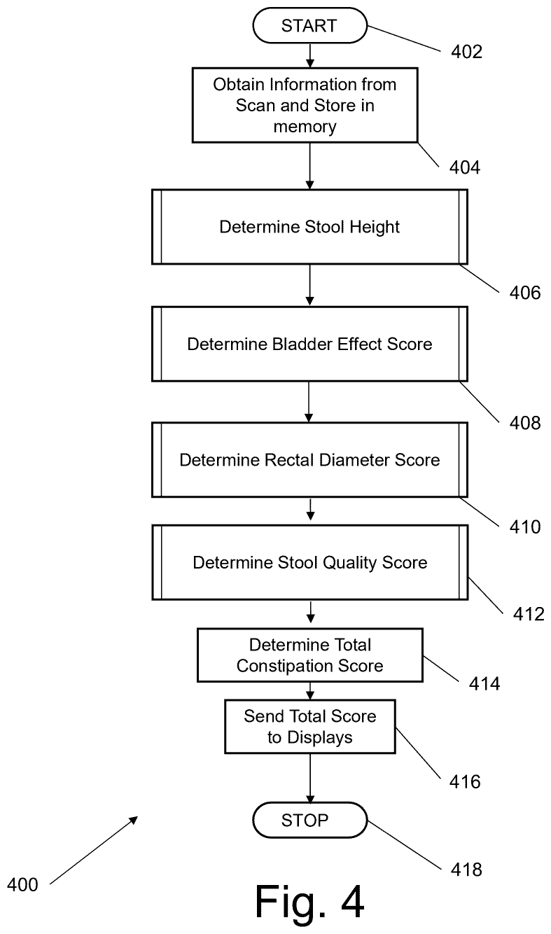

FIG. 4 illustrates an example embodiment of a process 400 that determines an overall constipation score. The process 400 may be executed by the central processor 106 described above or on another processor (not shown) in communication with the device 100. The process 400 begins at step 402 and flows to step 404 where data and or images are obtained and stored from the physical scanning process discussed above.

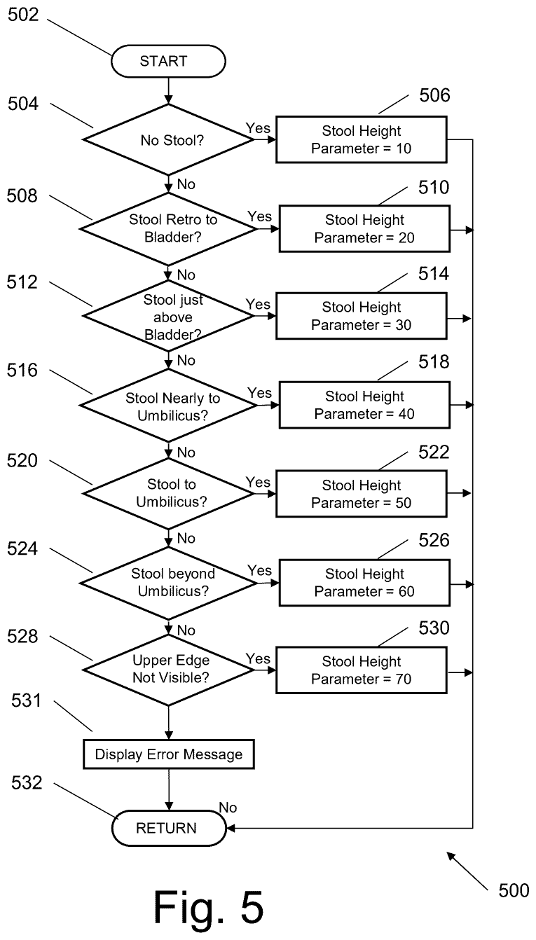

In step 406, the apex or stool height in the abdomen may be determined from the scanned data acquired previously and stored in the memory of the device or devices. In general, the apex is generally crescent-shaped. The location of the apex may be determined with the assistance of the AI model discussed above, and given a quantitative value representing the position within the abdomen as explained below in reference to FIG. 5.

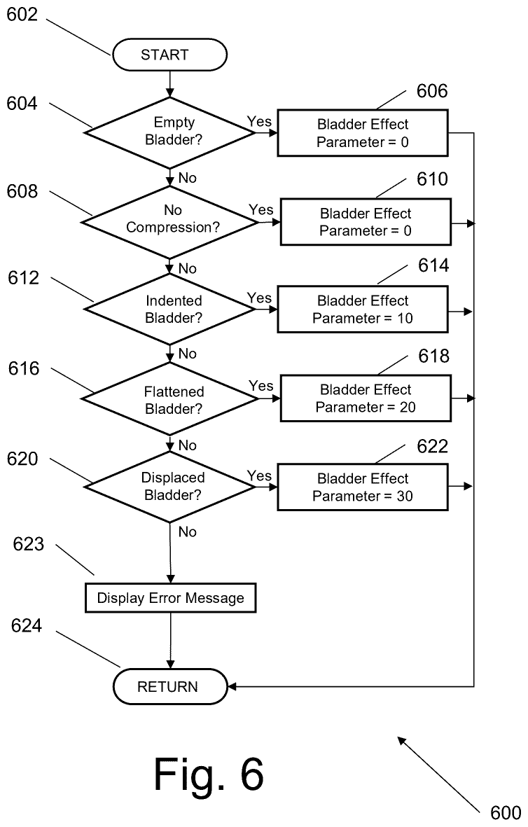

In step 408, the effects of the fecal mass on the bladder may be determined and given a score or quantitative value as explained in reference to FIG. 6 below.

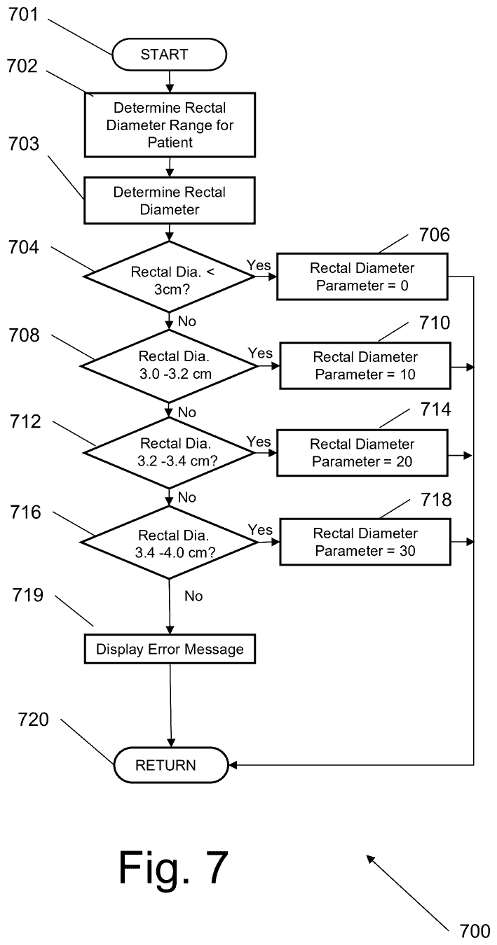

In step 410, a rectal diameter may be determined and quantified with the assistance of the AI model discussed above. One such example process for quantifying the rectal diameter effect is discussed below in reference to FIG. 7.

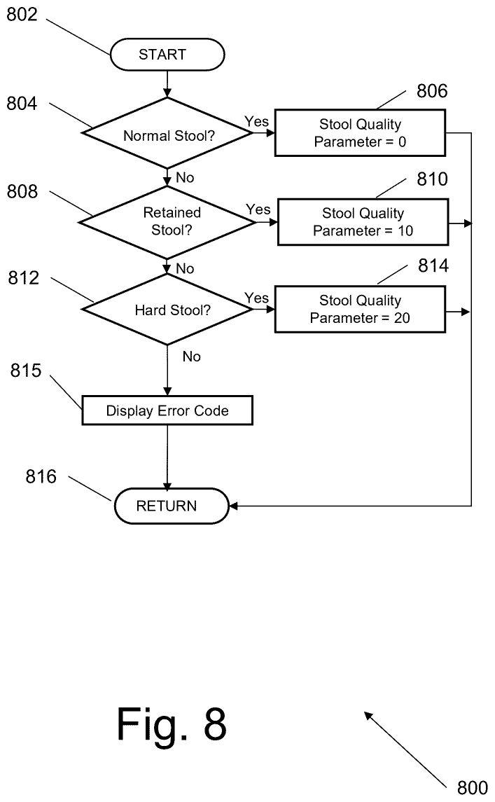

In step 412, the effects of stool quality are evaluated and given a quantitative value as explained below with reference to FIG. 8.

Once the apex height, the effects on the bladder, the rectal diameter, and stool quality have been quantified, in step 414 a processor then combines these factors to compute a total constipation score. In certain embodiments, the factors are weighted to represent the relative impact that each factor has relative to the other factors. For instance, if the stool height and bladder effect factors are more important than stool quality and rectal diameter factors, a weight is assigned to each factor. As an example, the total constipation score might be 1.5×(stool height)+1.7×(Bladder Effect)+(stool quality)+0.7×(rectal diameter)=constipation score. In certain embodiments, other factors learned via the AI learning process may also be considered and weighted. In step 416, this constipation score is then displayed on the relevant user interface (e.g. the display window 308 discussed above with respect to FIG. 3A or on a display of a computing device, such as a smartphone). This overall process 400 then stops at step 418.

### Illustrative Process for Determining a Stool Height Factor

FIG. 5 illustrates one example embodiment of a process 500 which determines a stool height score. It should be noted that the process 500 is only one method for determining a stool height score. Many other calculation methods could be used and many other numerical values may be assigned. All such methods are within the scope of the present invention.

The process starts at step 502 and continues to step 504, where a determination is made regarding whether the presence of stool is detected. If no stool is detected, the stool height parameter is set to a value representing this determination. For example, the value may be set to “10” in step 506. The process then flows to step 532 where the process 500 ends. Otherwise, the process flows to step 508.

At step 508, a determination is made regarding whether there is stool detected retro to or behind the bladder, if there is stool detected retro to the bladder, the stool height parameter is set to a value representing this determination. For example, the value may be set to “20” in step 510 and the process flows to step 532 where the process 500 ends. Otherwise, the process flows to step 512.

At step 512, a determination is made regarding whether there is stool detected above the bladder, if there is stool above the bladder, the stool height parameter is set to a value representing this determination. For example, the value may be set to “30” in step 514 and the process flows to step 532 where the process 500 ends. Otherwise, the process flows to step 516.

At step 516, a determination is made regarding whether there is stool detected nearly to the umbilicus, if there is stool detected nearly to the umbilicus, the stool height parameter is set to a value representing this determination. For example, the value may be set to “40” in step 518 and the process flows to step 532 where the process 500 ends. Otherwise, the process flows to step 520.

At step 520, a determination is made regarding whether there is stool detected in the umbilicus, if there is stool detected in the umbilicus, the stool height parameter is set to a value representing this determination. For example, the value may be set to “50” in step 522 and the process flows to step 532 where the process 500 ends. Otherwise, the process flows to step 524.

At step 524, a determination is made regarding whether there is stool detected beyond the umbilicus, if there is stool detected beyond the umbilicus, the stool height parameter is set to a value representing this determination. For example, the value may be set to “60” in step 526 and the process flows to step 532 where the process 500 ends. Otherwise, the process flows to step 528.

At step 528, a determination is made regarding whether the upper edge of the stool is beyond or outside of the range of the scanned images, if the upper edge of the stool is outside of the range of the scanned images, the stool height parameter is set to a value representing this determination. For example, the value may be set to “70” in step 530 and the process flows to step 532 where the process 500 ends. Otherwise, the process flows to step 531, where an error code is displayed so that the user knows that the stool position cannot be determined and that the scanning process described above may have to be repeated. The process then flows to step 532.

At step 532, the process 500 returns to the main process 400 where an overall constipation score is determined and displayed as discussed above.

### Illustrative Process for Determining a Bladder Effect Factor

FIG. 6 illustrates one example embodiment of a process 600 which determines a score or factor based on the effect the presence of stool may have on the bladder (e.g., the “bladder effect” score). It should be noted that the process 600 is only one method for determining a bladder effect score. Many other calculation methods could be used and many other numerical values may be assigned. All such methods are within the scope of the present invention.

The process starts at step 602 and continues to step 604, where a determination is made regarding whether the bladder is empty. If the bladder is empty, the bladder effect parameter is set to a value representing this determination. For example, the value may be set to “0” in step 606. The process then flows to step 624 where the process 600 ends. Otherwise, the process flows to step 608.

At step 608, a determination is made regarding whether the bladder is compressed by any stool, if the bladder is not compressed, the bladder effect parameter is set to a value representing this determination. For example, the value may be set to “0” in step 610 and the process flows to step 624 where the process 600 ends. Otherwise, the process flows to step 612.

At step 612, a determination is made regarding whether the bladder is indented by the presence of stool, if the bladder is indented, the bladder effect parameter is set to a value representing this determination. For example, the value may be set to “10” in step 614 and the process flows to step 624 where the process 600 ends. Otherwise, the process flows to step 616.

At step 616, a determination is made regarding whether the bladder is flattened by the presence of stool, if the bladder is flattened, the bladder effect parameter is set to a value representing this determination. For example, the value may be set to “20” in step 618 and the process flows to step 624 where the process 600 ends. Otherwise, the process flows to step 620.

At step 620, a determination is made regarding whether the bladder is displaced by the presence of stool, if the bladder is displaced, the bladder effect parameter is set to a value representing this determination. For example, the value may be set to “30” in step 622 and the process flows to step 624 where the process 600 ends. Otherwise, the process flows to step 623 where an error code is displayed so that the user knows that the process 600 cannot determine the bladder effect parameter. The process then flows to step 624.

At step 624, the process 600 returns to the main process 400 where an overall constipation score is determined and displayed as discussed above.

### Illustrative Process for Determining a Rectal Factor

FIG. 7 illustrates one example embodiment of a process 700 which determines a score or factor based on the diameter of the rectum (e.g., the “rectal diameter” parameter). A value of 2.5 cm or greater represents constipation in children less than four years of age. A rectal diameter of greater than 3 cm represents constipation in children greater than four years of age. It should be noted that the process 700 is only one method for determining a bladder effect score. Many other calculation methods could be used, and many other numerical values may be assigned. All such methods are within the scope of the present invention.

The process starts at step 701 and continues to step 702, where, based on the patient's age, various rectal diameter variables are retrieved from a database that correlates rectal diameters to an individual's age. For instance, for a particular age, the expected rectal diameter range may be 2 cm to 4.5 cm. After the expected range of variables has been retrieved, the process flows to step 703.

In step 703, the actual rectal diameter for the patient is determined from the ultrasound scans previously performed as described above. From step 703, the process moves to step 704, where the process determines if the rectal diameter is less than the expected variable, for instance, less than 3 cm. If the rectal diameter is less than 3 cm, the rectal diameter is set to a value representing this determination. For example, the rectal diameter parameter may be set to “0” in step 706. The process then flows to step 720 where the process 700 ends. Otherwise, the process flows to step 708.

At step 708, the process checks to determine if the rectal diameter is between 3.0 cm and 3.2 cm (as an example). If the rectal diameter is within the range of 3.0 cm to 3.2 cm, the rectal diameter is set to a value representing this determination. For example, the rectal diameter parameter may be set to “10” in step 710, and the process flows to step 720 where the process 700 ends. Otherwise, the process flows to step 712.

At step 712, the process checks to determine if the rectal diameter is between 3.2 cm and 3.4 cm. If the rectal diameter is within the range of 3.2 cm to 3.4 cm the rectal diameter is set to a value representing this determination. For example, the rectal diameter parameter may be set to “20” in step 714, and the process flows to step 720 where the process 700 ends. Otherwise, the process flows to step 716.

At step 716, the process checks to determine if the rectal diameter is between 3.4 cm and 4.0 cm. If the rectal diameter is within the range of 3.4 cm to 4.0 cm, the rectal diameter is set to a value representing this determination. For example, the rectal diameter parameter may be set to “30” step 718, and the process flows to step 720 where the process 700 ends. Otherwise, the process flows to step 719, where an error code is displayed so that the user knows that the process 700 cannot determine the rectal diameter or that the rectal diameter is outside of the expected range. The process then flows to step 720.

At step 720, the process 700 returns to the main process 400 where an overall constipation score is determined and displayed as discussed above.

### Illustrative Process for Determining a Stool Quality Factor

FIG. 8 illustrates one example embodiment of a process 800 which determines a score or factor based on the quality of the stool (e.g., the “stool quality” parameter). It should be noted that the process 800 is only one method for determining a bladder effect score. Many other calculation methods could be used and many other numerical values may be assigned. All such methods are within the scope of the present invention. The presence of feces of a normal quality may be seen as a half-moon-shaped hyperechoic area. In contrast, the presence of hard feces in the rectum is detected as a crescent-shaped acoustic shadow.

The process starts at step 802 and continues to step 804, where the process determines if the stool has a normal quality as determined by the shape of the shadow. If the stool is determined to have normal quality, the stool quality parameter is set to a value representing this determination. For example, the stool quality parameter may be set to “0” in step 806. The process then flows to step 816 where the process 800 ends. Otherwise, the process flows to step 808.

At step 808, the process checks to determine if the stool is retained (e.g., has a crescent-shaped acoustic shadow). If the stool is retained, the stool quality parameter is set to a value representing this determination. For example, the stool quality parameter may be set to “10” in step 810, and the process flows to step 816 where the process 800 ends. Otherwise, the process flows to step 812.

At step 812, the process checks to determine if the stool is hard. If the stool is hard, the stool quality parameter is set to a value representing this determination. For example, the stool quality parameter may be set to “20” in step 814 and the process flows to step 816 where the process 800 ends. Otherwise, the process flows to step 815, which displays an error code so that the user knows that the stool quality parameter cannot be determined. The process then flows to step 816.

At step 816, the process 800 returns to the main process 400 where an overall constipation score is determined and displayed as discussed above.

In some embodiments, the processed data is converted into image data and a constipation score is computed as described above. In certain embodiments, the data may be stored internally and/or transferred (via wired or wireless connectivity) to a cloud or external storage, or another computer device for display on an associated screen via an app. The overall constipation score is then displayed on the display window of the device itself, or in some embodiments, on a separate computer device.

The abstract of the disclosure is provided for the sole reason of complying with the rules requiring an abstract, which will allow a searcher to quickly ascertain the subject matter of the technical disclosure of any patent issued from this disclosure. It is submitted with the understanding that it will not be used to interpret or limit the scope or meaning of the claims.

For the U.S. application: Any advantages and benefits described may not apply to all embodiments of the invention. When the word “means” is recited in a claim element, Applicant intends for the claim element to fall under 35 USC 112(f). Often a label of one or more words precedes the word “means”. The word or words preceding the word “means” is a label intended to ease referencing of claims elements and is not intended to convey a structural limitation. Such means-plus-function claims are intended to cover not only the structures described herein for performing the function and their structural equivalents but also equivalent structures. For example, although a nail and a screw have different structures, they are equivalent structures since they both perform the function of fastening. Claims that do not use the word “means” are not intended to fall under 35 USC 112(f).

The foregoing description of the embodiments of the invention has been presented for the purposes of illustration and description. It is not intended to be exhaustive or to limit the invention to the precise form disclosed. Many combinations, modifications, and variations are possible in light of the above teaching. For instance, in certain embodiments, each of the above-described components and features may be individually or sequentially combined with other components or features and still be within the scope of the present invention. Undescribed embodiments which have interchanged components are still within the scope of the present invention. It is intended that the scope of the invention be limited not by this detailed description, but rather by the claims.

1. A medical ultrasound device, comprising:
a power source,
a control unit coupled to the power source;
a display in electrical communication with the control unit;
a transducer in communication with the control unit;
a memory in electric communication with the control unit, the memory having instructions for:
recording ultrasound data generated during a scanning session;
determining a stool height effect parameter of a patient from the data generated during the scanning session;
determining a patient's bladder effect parameter from the data generated during the scanning session;
determining a patient's rectal diameter effect parameter from the data generated during the scanning session;
determining a patient's stool quality effect parameter from data generated during the scanning session;
determining an overall constipation score for the patient using the stool height effect parameter, the bladder effect parameter, the rectal diameter effect parameter, and the stool quality effect parameter;
displaying the overall constipation score on the display, and
wherein the stool height effect parameter, the bladder effect parameter, the rectal diameter effect parameter, and the stool quality effect parameter are assigned different individual weights when determining the overall constipation score to account for the impact that each parameter has relative to the other parameters.

2. (canceled)

3. The medical ultrasound device of claim 1, wherein the stool height effect parameter is determined by assigning a value based on a position of the stool with respect to the patient's bladder and umbilicus.

4. The medical ultrasound device of claim 1, wherein the bladder effect parameter is determined by assigning a value based on a state of the bladder.

5. The medical ultrasound device of claim 4, wherein the state of the bladder is selected from the group consisting of an empty bladder, a non-compressed bladder, an indented bladder, and a displaced bladder.

6. The medical ultrasound device of claim 1, wherein the stool quality effect parameter is determined by assigning a value based on whether the stool is normal, the stool is retained, or the stool is hard.

7. The medical ultrasound device of claim 1, wherein the control unit is in communication with an artificial intelligence system comprising a neural network configured for image recognition of anatomical structures within a human torso.

8-15. (canceled)
